(19) United States
(12) Patent Application Publication
(10) Pub. No.: US 2006/0160116 A1
(43) Pub. Date: Jul. 20, 2006

(54) Repetitive sequence-free DNA libraries

(75) Inventors: Allen T. Christian, Middleton, WI (US); Larry C. Dugan, Modesto, CA (US); Joel Bedford, Fort Collins, CO (US)

Correspondence Address: Eddie E. Scott;Assistant Laboratory Counsel, Lawrence Livermore National Laboratory, P.O. Box 808, L-703, Livermore, CA, 94551, US

(73) Assignee: The Regents of the University of California

(21) Appl. No.: 11/299,347

(22) Filed: Dec. 8, 2005

Related U.S. Application Data
(60) Provisional application No. 60/637,367, filed on Dec. 16, 2004.

(51) Int. Cl. C40B 40/08; C12Q 1/68

(52) U.S. Cl. 435/6; 435/91.2

(57) ABSTRACT

A method of creating a repetitive sequence-free DNA library comprising the steps of providing a DNA library, providing an amplification mixture from the DNA library, and adding a repetitive sequence fraction DNA to the amplification mixture to produce the repetitive sequence-free DNA library. The invention also provides a method of creating a whole chromosome painting probe comprising the steps of providing a DNA library, providing an amplification mixture from the DNA library, adding a repetitive sequence fraction DNA to the amplification mixture to produce the repetitive sequence-free DNA library, and labeling the repetitive sequence-free DNA library to produce the whole chromosome painting probe. The invention also provides a method of in-situ hybridization comprising the steps of providing a DNA library, providing an amplification mixture from the DNA library, adding a repetitive sequence fraction DNA to the amplification mixture to produce the repetitive sequence-free DNA library, labeling the repetitive sequence-free DNA library to produce the whole chromosome painting probe, and using the painting probe in in-situ hybridization.

### CROSS-REFERENCE TO RELATED APPLICATIONS

This application claims the benefit of U.S. Provisional Patent Application No. 60/637,367 filed Dec. 16, 2004 and titled “Repetitive Sequence-free DNA Libraries.” U.S. Provisional Patent Application No. 60/637,367 filed Dec. 16, 2004 and titled “Repetitive Sequence-free DNA Libraries” is incorporated herein by this reference.

The United States Government has rights in this invention pursuant to Contract No. W-7405-ENG-48 between the United States Department of Energy and the University of California for the operation of Lawrence Livermore National Laboratory.

### BACKGROUND

1. Field of Endeavor

The present invention relates to DNA libraries and more particularly to repetitive sequence-free DNA Libraries.

2. State of Technology

U.S. Pat. No. 6,841,347 for in vivo construction of DNA libraries issued Jan. 11, 2005 to Anntonis Aervos provides the following state of technology information, “A cDNA library is a collection of cloned DNA molecules propagated in an appropriate host. It is usually derived from the mRNA population of a particular cell, tissue or organ by reverse transcription, cloned into a vector molecule and propagated in an appropriate host cell. cDNA libraries are useful in numerous applications. cDNA libraries can be used to isolate and identify cell-specific expressed sequences. A cDNA clone isolated from a library can be sequenced and translated (e.g., by computer programs) to derive the primary amino acid sequence of the encoded protein or can be used as a labeled probe to investigate gene expression in vivo. cDNA libraries can also be used in a two-hybrid assay to screen a large number of candidate proteins and identify those which interact with a particular target protein. In this approach, cDNAs are incorporated into activation domain vectors to provide random proteins fused to an activation domain of a known transcription factor. Vectors encoding the target protein fused to the DNA binding domain of the transcription factor, and the library of activation domain hybrids are cotransformed into a reporter strain. Interaction of the target protein moiety of a target protein DNA binding domain fusion protein with a protein encoded by cDNA brings the DNA binding domain into proximity with the activation domain fused to the cDNA encoded protein. The resulting transcription identifies a positive clone. Once a positive clone has been identified, the gene corresponding to the interacting protein can be isolated and analyzed.”

### SUMMARY

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

DNA libraries are used daily, in research laboratories and hospitals, as probes to locate abnormalities in chromosomes. Many birth defects such as Down's Syndrome and diseases like cancer are detected using fluorescently-labeled DNA probes made from these libraries. These probes can be made specific to particular chromosomes, or even to regions within chromosomes. However, to use these probes requires the co-hybridization of unlabeled DNA to block the repetitive elements of DNA in the probes. Without this addition, the probe is non-specific and will bind to every chromosome in the cell. The ability to make libraries that are free of these repetitive elements, and thus do not require blocking DNA to be added to the reactions, would represent a significant savings in cost for a research laboratory.

The present invention provides a system for making such libraries that holds for any species of animal. The present invention provides a method of creating a repetitive sequence-free DNA library. The method comprises the steps of providing a DNA library, providing an amplification mixture from the DNA library, and adding a repetitive sequence fraction DNA to the amplification mixture to produce the repetitive sequence-free DNA library. The present invention also provides a method of creating a whole chromosome painting probe. The method comprises the steps of providing a DNA library, providing an amplification mixture from the DNA library, adding a repetitive sequence fraction DNA to the amplification mixture to produce the repetitive sequence-free DNA library, and labeling the repetitive sequence-free DNA library to produce the whole chromosome painting probe. The present invention also provides a method of in-situ hybridization. The method comprises the steps of providing a DNA library, providing an amplification mixture from the DNA library, adding a repetitive sequence fraction DNA to the amplification mixture to produce the repetitive sequence-free DNA library, labeling the repetitive sequence-free DNA library to produce the whole chromosome painting probe, and using the painting probe in in-situ hybridization.

The present invention provides a simple method for producing significant volumes of chromosome-specific painting probes. The entire process can be done in less than one day and yields probes with high specificity without the use of additional competitor DNA. The present invention has use in research and in hospitals. Cytogenetic analysis is an important diagnostic tool in prenatal care, as well as oncology. These laboratories are set up to require processes that are as simple and foolproof as possible. To remove an element of the in situ hybridization process, as well as decreasing the cost of the probes, will represent a significant improvement over existing technology.

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1 illustrates one embodiment of a method of creating a repetitive sequence-free DNA library of the present invention.

FIG. 2 illustrates another embodiment of a method of creating a whole chromosome painting probe of the present invention.

FIG. 3 illustrates another embodiment of a method of in-situ hybridization of the present invention.

FIG. 4 illustrates another embodiment of a method of creating a repetitive sequence-free DNA library of the present invention.

FIG. 5 illustrates another embodiment of a method of creating a whole chromosome painting probe of the present invention.

FIG. 6 illustrates another embodiment of a method of in-situ hybridization of the present invention.

### DETAILED DESCRIPTION OF THE INVENTION

Referring to the drawings, to the following detailed description, and to incorporated materials, detailed information about the invention is provided including the description of specific embodiments. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

Modern cytogenetic techniques including fluorescence in situ hybridization (FISH), comparative genomic hybridization (CGH), Multiplex-FISH (M-FISH) and Spectral karyotyping (SKY), have become extensively used techniques in both diagnostic and research laboratories. The probes used in these techniques contain both unique and repetitive sequences, which bind to target DNA. The repetitive sequences are suppressed from binding to the target by the addition of competitive blocking DNA, usually Cot-1 DNA. This process requires large amounts of Cot-1 DNA, often 20-50-fold excesses, that is expensive when purchased commercially.

Recent publications have described a method for removing repetitive sequences using affinity chromatography producing PCR-amplifiable, chromosome-specific painting probes. This method solves the problem of having to use high levels of expensive Cot-1 DNA, but is difficult and time consuming. It requires multiple PCR amplifications and affinity chromatography purifications for many of the probes. Furthermore, the initial outlay for materials can be rather expensive.

Applicants have developed a method to suppress the PCR amplification of repetitive sequences in whole chromosome painting probes by adding Cot-1 DNA to the amplification mixture. The repetitive sequences in the Cot-1 DNA bind to their homologous sequences in the probe library, prevent the binding of primers, and interfere with extension of the probe sequences, greatly decreasing PCR efficiency selectively across these blocked regions. A second labeling reaction is then done and this product is resuspended in FISH hybridization mixture without further addition of blocking DNA. The hybridization produces little if any non-specific binding on any other chromosomes. Applicants have been able to successfully use this procedure with both human and rat chromosome probes. This technique should be applicable in producing probes for CGH, M-FISH and SKY, as well as reducing the presence of repetitive DNA in genomic libraries.

The present invention provides a simple method for producing significant volumes of chromosome-specific painting probes. The entire process can be done in less than one day and yields probes with high specificity without the use of additional competitor DNA. Additional details of the present invention are described in the article “Polymerase Chain Reaction-based Suppression of Repetitive Sequences in Whole Chromosome Painting Probes for FISH” by Lawrence C. Dugan, Melissa S. Pattee, Jennifer Williams, Mike Eklund, J. Karen Sorensen, Joel S. Bedford and Allen T. Christian, in Chromosome Research, 13 (1), p 27-32 (2005). The article and all figures, data, and information “Polymerase Chain Reaction-based Suppression of Repetitive Sequences in Whole Chromosome Painting Probes for FISH” by Lawrence C. Dugan, Melissa S. Pattee, Jennifer Williams, Mike Eklund, J. Karen Sorensen, Joel S. Bedford and Allen T. Christian, in Chromosome Research, 13 (1), p 27-32 (2005) is incorporated herein by reference. A copy of the article is enclosed in a Prior Art statement accompanying this application.

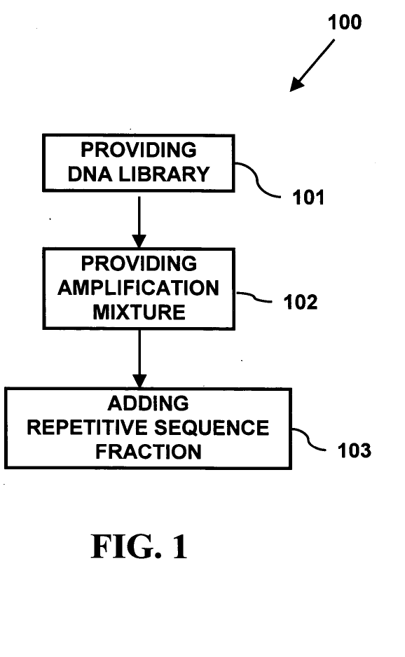

Referring now to the drawings, FIG. 1 illustrates one embodiment of the present invention. This embodiment is designated generally by the reference numeral 100. The system 100 provides method of creating a repetitive sequence-free DNA library. The system 100 comprises a number of steps. Step 101 comprises providing a DNA library. Step 102 comprises providing an amplification mixture from the DNA library. Step 103 comprises adding a repetitive sequence fraction DNA to the amplification mixture to produce the repetitive sequence-free DNA library. In one embodiment, the step of adding a repetitive sequence fraction to the amplification mixture comprises adding Cot-1 DNA to the amplification mixture. In another embodiment, the step of adding a repetitive sequence fraction to the amplification mixture comprises adding dideoxyhucleotide triphosphate terminated Cot-1 DNA to said amplification mixture. In another embodiment, the step of adding a repetitive sequence fraction to the amplification mixture comprises adding hybloc competitor DNA to the amplification mixture.

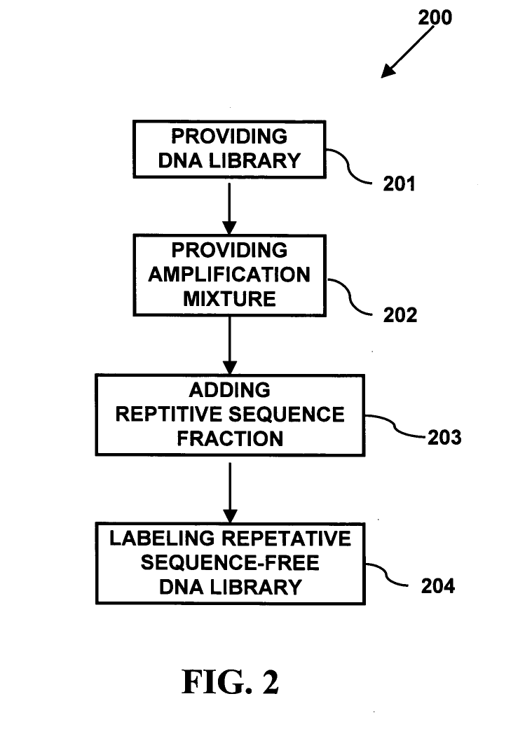

Referring to FIG. 2 another embodiment of the present invention is illustrated. This embodiment is designated generally by the reference numeral 200. The system 200 provides method of creating a whole chromosome painting probe. The system 200 comprises a number of steps. Step 201 comprises providing a DNA library. Step 202 comprises providing an amplification mixture from the DNA library. Step 203 comprises adding a repetitive sequence fraction DNA to the amplification mixture to produce a repetitive sequence-free DNA library. Step 204 comprises labeling the repetitive sequence-free DNA library to produce the whole chromosome painting probe. In one embodiment, the step of adding a repetitive sequence fraction to the amplification mixture comprises adding Cot-1 DNA to the amplification mixture. In another embodiment, the step of adding a repetitive sequence fraction to the amplification mixture comprises adding dideoxynucleotide triphosphate terminated Cot-1 DNA to said amplification mixture. In another embodiment, the step of adding a repetitive sequence fraction to the amplification mixture comprises adding hybloc competitor DNA to the amplification mixture.

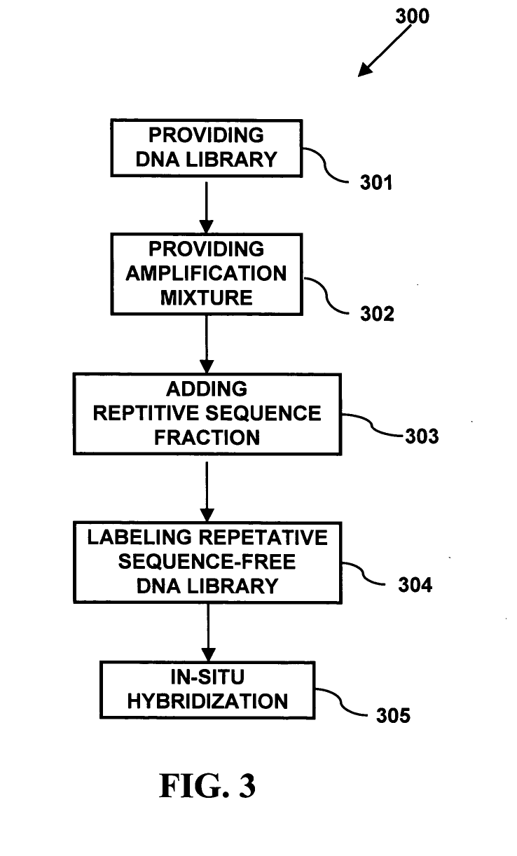

Referring now to FIG. 3 another embodiment of the present invention is illustrated. This embodiment is designated generally by the reference numeral 300. The system 300 provides method of in-situ hybridization. The system 300 comprises a number of steps. Step 301 comprises providing a DNA library. Step 302 comprises providing an amplification mixture from the DNA library. Step 303 comprises adding a repetitive sequence fraction DNA to the amplification mixture to produce a repetitive sequence-free DNA library. Step 304 comprises labeling the repetitive sequence-free DNA library to produce the whole chromosome painting probe. Step 305 comprises using the painting probe in in-situ hybridization. In one embodiment, the hybridization mixture is a FISH hybridization mixture. In one embodiment, the hybridization mixture is an M-FISH hybridization mixture. In one embodiment, the hybridization mixture is a SKY hybridization mixture. In one embodiment, the hybridization mixture is a CGH hybridization mixture. In one embodiment, the step of adding a repetitive sequence fraction to the amplification mixture comprises adding Cot-1 DNA to the amplification mixture. In another embodiment, the step of adding a repetitive sequence fraction to the amplification mixture comprises adding dideoxynucleotide triphosphate terminated Cot-1 DNA to said amplification mixture. In another embodiment, the step of adding a repetitive sequence fraction to the amplification mixture comprises adding dideoxynucleotide triphosphate terminated Cot-1 DNA to said amplification mixture. In another embodiment, the step of adding a repetitive sequence fraction to the amplification mixture comprises adding hybloc competitor DNA to the amplification mixture. In another embodiment, blocking DNA is not used in subsequent amplification mixtures. In another embodiment, the step of adding a repetitive sequence fraction DNA to the amplification mixture comprises using a standard PCR amplification procedure with the addition of 1 mg of species-specific Cot-1 DNA in the amplification mixture.

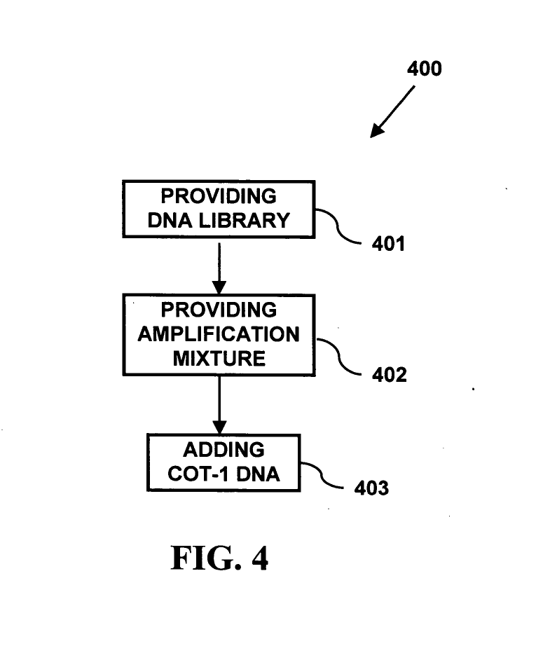

Referring to the drawings, FIG. 4 illustrates another embodiment of the present invention. This embodiment is designated generally by the reference numeral 400. The system 400 provides method of creating a repetitive sequence-free DNA library. The system 400 comprises a number of steps. Step 401 comprises providing a DNA library. Step 402 comprises providing an amplification mixture from the DNA library. Step 403 comprises adding a repetitive sequence fraction DNA to the amplification mixture to produce the repetitive sequence-free DNA library. The step 403 of adding a repetitive sequence fraction to the amplification mixture comprises adding Cot-1 DNA to the amplification mixture.

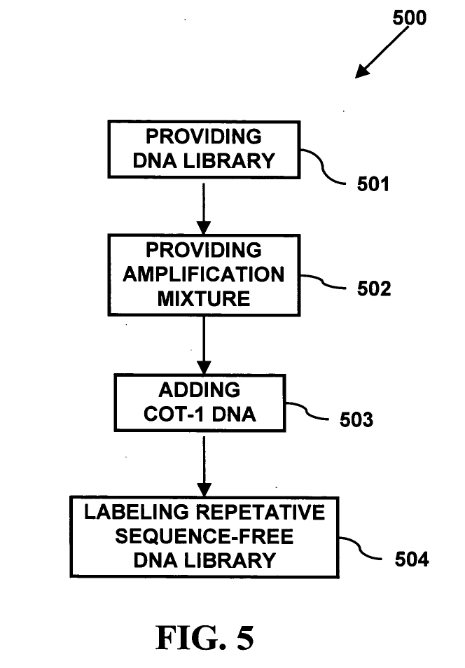

Referring to FIG. 5 another embodiment of the present invention is illustrated. This embodiment is designated generally by the reference numeral 500. The system 500 provides method of creating a whole chromosome painting probe. The system 500 comprises a number of steps. Step 501 comprises providing a DNA library. Step 502 comprises providing an amplification mixture from the DNA library. Step 503 comprises adding a repetitive sequence fraction DNA to the amplification mixture to produce a repetitive sequence-free DNA library. The step 503 of adding a repetitive sequence fraction to the amplification mixture comprises adding Cot-1 DNA to the amplification mixture. Step 504 comprises labeling the repetitive sequence-free DNA library to produce the whole chromosome painting probe.

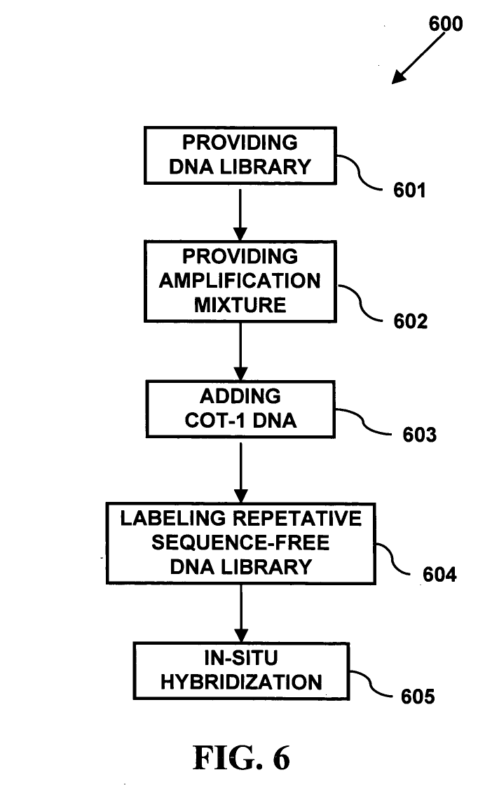

Referring now to FIG. 6 another embodiment of the present invention is illustrated. This embodiment is designated generally by the reference numeral 600. The system 600 provides method of in-situ hybridization. The system 600 comprises a number of steps. Step 601 comprises providing a DNA library. Step 602 comprises providing an amplification mixture from the DNA library. Step 603 comprises adding a repetitive sequence fraction DNA to the amplification mixture to produce a repetitive sequence-free DNA library. The step 603 of adding a repetitive sequence fraction to the amplification mixture comprises adding Cot-1 DNA to the amplification mixture. Step 604 comprises labeling the repetitive sequence-free DNA library to produce the whole chromosome painting probe. Step 605 comprises using the painting probe in in-situ hybridization. In one embodiment, the hybridization mixture is a FISH hybridization mixture. In one embodiment, the hybridization mixture is an M-FISH hybridization mixture. In one embodiment, the hybridization mixture is a SKY hybridization mixture. In one embodiment, the hybridization mixture is a CGH hybridization mixture. In another embodiment, blocking DNA is not used in subsequent amplification mixtures. In another embodiment, the step of adding a repetitive sequence fraction DNA to the amplification mixture comprises using a standard PCR amplification procedure with the addition of 1 mg of species-specific Cot-1 DNA in the amplification mixture.

Standard Library Amplification and Labeling by PCR

A 15 μl reaction volume was prepared using 200 ng chromosome specific library, 1.51 μl Thermosequenase reaction buffer (USB, Cleveland, Ohio), 1.5 μl 10×dNTP solution (Roche Molecular, Indianapolis, Ind.) (80 μM final concentration) 0.6 μl 100 μM Telenius primer (5′-CCGACTCGAGNNNNNNATGTGG-3′) (MacroMolecular Resources, Fort Collins, Colo.), 6U Thermosequenase polymerase (USB, Cleveland, Ohio) and distilled, diionized H₂O. The libraries were amplified using a MJResearch PT-100 thermocycler (MJResearch, Watertown, Mass.). The reaction profile used was as follows; 95° C. for 5min. followed by 15 cycles of 94° C. for 1 min., 60° C. for 1 min. and 72° C. for 3 min. This was followed by 5 min. at 72° C. and a hold at 4° C. until tubes were removed. Products were then purified using Qiagen's Qiaquick PCR purification kit and 3 μl were run on a 1.5% agarose gel against size and concentration standards for 60 minutes at 100V. Products should be in the range of 300-800 bp in size and a concentration of 100-200 ng/μl. 3 μl of the product was diluted 1:10 in 10 mM Tris-HCl, pH 8.5. A labeling step was then performed in a 50 μl volume containing 1 μl of diluted chromosome library (˜10-20 ng) from the above reaction, 5 μl Thermosequenase reaction buffer (USB, Cleveland, Ohio), 5 μl of 10×dNTP solution (Roche Molecular, Indianapolis, Ind.) (80 μM final concentration), 2 μl of 100 μM Telenius primer, 0.5 μl AmpliTaq LD polymerase, 2 μl of digoxigenin-11-dUTP and distilled, deionized H₂O. The same reaction profile was used as above. This product was not further purified.

Repetitive-sequence Depletion by PCR Using Cot-1 DNA

The standard PCR amplification procedure was used with the addition of 1 mg of species-specific Cot-1 DNA in the amplification reaction. The result of this protocol is a DNA library that does not require that any blocking DNA be used in subsequent in situ hybridization experiments.

Applicants' invention provides a simple method to produce virtually unlimited quantities of Cot-1 depleted whole chromosome-specific painting probes (WCPs). The entire process can be done in less than one day and yields probes with high specificity without the use of additional competitor DNA. Human chromosome X and rat chromosome 1 specific probes were prepared by microdissection of normal metaphase chromosomes. The microdissected chromosomes were then amplified using the degenerate oligonucleotide-primed PCR protocol (DOP-PCR). This protocol uses a single primer containing a degenerate 6-nucleotide sequence and an initial ramping step to randomly incorporate the primer into target DNA. Once incorporated, target DNA can be further amplified with this same single primer. WCPs produced in this manner are complex enough to provide continuous coverage of target chromosomes. The WCPs produced here were tested against traditional hybridization cocktails on normal human fibroblast and rat lymphocyte metaphase spreads.

Materials and Methods

Chromosome libraries—Chromosome X was kindly provided by Dr. Maria Muhlmann-Diaz, formerly of Colorado State University and was produced using traditional microdissection techniques. Rat chromosome 1 library (RNO1) was also prepared using standard microdissection techniques. Briefly, metaphase spreads are dropped onto glass coverslips and air-dried. Chromosomes are identified under phase-contrast, transmitted light on an inverted microscope, often with the aid of G-banding. A glass needle attached to a micromanipulator is then used to scrape desired chromosomes from the coverslip, one at a time. The chromosome DNA is then transferred to a PCR tube by breaking off the tip of the needle containing the DNA in the tube. Multiple copies of a single chromosome are usually collected in the same tube. Collection of multiple copies of the same chromosome improves complexity and coverage of the probe on target chromosomes. The tubes were then centrifuged and loaded with PCR reaction solution. The PCR reaction solution contains 1.5 μl Thermosequenase reaction buffer, 1.5 μl 10×dNTP solution, (200 mM dATP, dTTP, dCTP, dGTP), 0.6 μl of 100 μM DOP primer (5′-CCGACTCGAGNNNNNNATGTGG-3′), 6 U Thermosequenase polymerase and distilled, deionized H₂O to a final volume of 15 μl. The microdissected chromosomes were amplified using an MJResearch PT-ioo thermocycler. The temperature-time reaction cycling profile used was as follows; 95° C. for 10 min, 8 cycles at 94° C. for 1 min, 30° C. for 5 min, and a ramp of 0.1° C./s to 65° C. for 5 min, followed by 12 cycles of 94° C. for 1 min, 56° C. for 1 min and 72° C. for 5 min. This was followed by 5 min at 72° C. and a hold at 4° C. until tubes were removed.

The standard amplification procedure described above was used with the inclusion of 1 μg human Cot-1 DNA for human Chromosome X and 1 μg rat Cot-1 DNA in the amplification reaction. Prior to use, the Cot-1 DNA was ethanol precipitated and resuspended at 1 μg/μl in 10 mM Tris-HCl, pH 8.5, to remove EDTA, which inhibits PCR amplification.

Metaphase Chromosome Preparation

Human metaphase spreads were prepared by growing human BJ1 cells (ATCC) to confluence in DMEM/F12 supplemented with 10% fetal bovine serum (FBS). The cells were then subcultured at a 1:5 dilution and incubated for 30-36 hours. Mitotic cells were collected and dropped on slides as previously described.

Rat metaphase spreads were prepared from blood cultures obtained by cardiac puncture of 8-12 week old, male Sprague-Dawley rats following the procedure in with minor modifications. Animals were housed in LLNL Animal Care Facility. Fresh blood was washed 2× with 5 ml RPMI 1640 media containing 10 U/ml Heparin (Sigma) and incubated in RPMI-1640 supplemented with 10% FBS (Sigma), 3 μg/ml Concanavalin A (Sigma), 100 μg/ml Lipopolysaccharide (Sigma), 1% L-glutamine (Gibco) and Antibiotic/antimycotic (Gibco). Cultures were incubated for 62 h at 37° C. in a humidified incubator containing 5% CO₂. Colcemid (Gibco) was then added at a final concentration of 0.1 μg/ml for 4 h. Mitotic cells were then collected and dropped on slides as previously described.

Fluorescence in situ Hybridization

A 4 μl volume of Alexa Fluor-488 labeled library containing 200 ng/ul was diluted with or without 4 μg Cot-1 DNA in a final volume of 15 μl containing 50% formamide, 2×SSC and 10% Dextran sulfate. The probe cocktail was then denatured for 10 min. at 84° C. and incubated at 37° C. for 45-60 min. Target slides were prepared by dehydration in an ethanol series consisting of 2 min. washes in 70%, 85% and 100% ethanol at room temperature. Slides were air-dried and denatured for 2-3 min. in 70% formamide, 30% 2×SSC@72° C. This was followed by a second dehydration series. The denaturations were timed so as to be completed simultaneously. The probe cocktail was then placed on the target slide and covered with a 22×22 mm coverslip. The coverslip was sealed with rubber cement and the slide was placed in a sealed slide box and incubated for 1-2 days at 37° C.

After incubation, the rubber cement and coverslip were carefully removed and the slide was rinsed 2× in 50% formamide, 2×SSC at 45° C. for 5 min per rinse. Slides were then rinsed 2× in 2×SSC at 45° C. for 5 min per rinse. This was followed by rinsing 2× in room temperature 1×PN buffer for 3 min per rinse. Finally, 10 μl of antifade solution containing 2.5 ng/μl DAPI counterstain was placed on the target area and covered with a coverslip.

Image Capture and Analysis

Slides were imaged using a Zeiss Axioskop microscope equipped with epifluorescence and standard DAPI/FITC/Texas red excitation filters and a triple bandpass DAPI/FITC/Texas red filter set. Images were captured using a Photometrics' SenSys CCD camera and Applied Imaging's Quips image analysis software.

Results and Discussion

As expected, the unblocked presence of labeled, repetitive sequences common to all chromosomes results in the more or less uniform painting of all chromosomes. FIG. 1B in the article, “Polymerase Chain Reaction-based Suppression of Repetitive Sequences in Whole Chromosome Painting Probes for FISH” by Lawrence C. Dugan, Melissa S. Pattee, Jennifer Williams, Mike Eklund, J. Karen Sorensen, Joel S. Bedford and Allen T. Christian, in Chromosome Research, 13 (1), p 27-32 (2005) shows results of hybridization of the chromosome X library amplified under standard conditions, without Cot-1 DNA in the PCR reaction, but in this case hybridized in the usual way with the addition of a 20-fold excess of unlabeled, blocking Cot-1 DNA during probe hybridization on the slide. The presence of the unlabelled Cot-1 DNA competitively blocks painting of all but the X chromosome. FIG. 1C of the article shows the results obtained following hybridization of the chromosome X library amplified with Cot-1 DNA present in the PCR reaction, but without addition of unlabeled Cot-1 DNA during the hybridization. Thus, the blocking occurred by competitive hybridization during the PCR reaction, rather than on the slide.

This method can be expanded for use in other non-human mammalian species as shown in FIG. 2 of the article. A whole chromosome probe for RNO1 was prepared by microdissection, labeled and hybridized to rat metaphase spreads in the presence of rat Cot-1 DNA. As seen in FIG. 2A of the article, this produces little to no background signal and high specificity to chromosome 1. By amplifying the chromosome library in the presence of rat Cot-1 DNA, followed by labeling and hybridization without additional Cot-1 DNA, Applicants were able to obtain similar results as the standard procedure, FIG. 2B of the article.

The addition of a 10-fold excess of Cot-1 DNA to a PCR amplification reaction involving chromosome-specific libraries blocks, or at least drastically reduces the PCR efficiency of the amplification of the highly repetitive sequences in the library while allowing for the unimpeded amplification of unique sequences. It is thought that the presence of the Cot-1 DNA in the PCR reaction binds competitively with the DOP-primer to repetitive elements during the annealing step, minimizing the amplification of these repetitive elements. As shown in FIGS. 1 and 2 of the article, this technique yields hybridizations of probes of equal quality to standard procedures utilizing large quantities of Cot-1 DNA. The value of this approach lies in the fact that, once made, Cot-1 free libraries remain so, and no repetitive element blocking is ever necessary in subsequent reactions.

Applicants have presented a method for removing repetitive sequences from chromosome-specific libraries that is quick, inexpensive and produces results equaling traditional FISH methods. Applicants have estimated that for each initial 15 μl PCR-blocking reaction, Applicants can produce >1000×10 μl hybridizations without additional Cot-1 DNA. At 20 μl Cot-1/hybridization and a cost of −$100/500 μl Cot-1DNA, this comes to a savings of >$5000.

Using this technique, a large volume of a chromosome specific DNA library can be generated in a single day. This method should be applicable to any process where the need exists for high quality libraries of unique or low-copy DNA sequences.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

The invention claimed is:

1. A method of creating a repetitive sequence-free DNA library, comprising the steps of:
providing a DNA library,
providing an amplification mixture from said DNA library, and
adding a repetitive sequence fraction DNA to said amplification mixture to produce the repetitive sequence-free DNA library.

2. The method of creating a repetitive sequence-free DNA library of claim 1 wherein said step of adding a repetitive sequence fraction to said amplification mixture comprises adding Cot-1 DNA to said amplification mixture.

3. The method of creating a repetitive sequence-free DNA library of claim 1 wherein said step of adding a repetitive sequence fraction to said amplification mixture comprises adding dideoxynucleotide triphosphate terminated Cot-1 DNA to said amplification mixture.

4. The method of creating a repetitive sequence-free DNA library of claim 1 wherein said step of adding a repetitive sequence fraction to said amplification mixture comprises adding hybloc competitor DNA to said amplification mixture.

5. A method of creating a whole chromosome painting probe, comprising the steps of:
providing a DNA library,
providing an amplification mixture from said DNA library,
adding a repetitive sequence fraction DNA to said amplification mixture to produce the repetitive sequence-free DNA library, and
labeling said repetitive sequence-free DNA library to produce the whole chromosome painting probe.

6. The method of creating a whole chromosome painting probe of claim 5 wherein said step of adding a repetitive sequence fraction to said amplification mixture comprises adding Cot-1 DNA to said amplification mixture.

7. The method of creating a whole chromosome painting probe of claim 5 wherein said step of adding a repetitive sequence fraction to said amplification mixture comprises adding dideoxynucleotide triphosphate terminated Cot-1 DNA to said amplification mixture.

8. The method of creating a whole chromosome painting probe of claim 5 wherein said step of adding a repetitive sequence fraction to said amplification mixture comprises adding hybloc competitor DNA to said amplification mixture.

9. A method of in-situ hybridization, comprising the steps of:
providing a DNA library,
providing an amplification mixture from said DNA library,
adding a repetitive sequence fraction DNA to said amplification mixture to produce the repetitive sequence-free DNA library,
labeling said repetitive sequence-free DNA library to produce the whole chromosome painting probe, and
using said painting probe in in-situ hybridization.

10. The method of in-situ hybridization of claim 9 wherein said step of adding a repetitive sequence fraction to said amplification mixture comprises adding Cot-1 DNA to said amplification mixture.

11. The method of in-situ hybridization of claim 9 wherein said step of adding a repetitive sequence fraction to said amplification mixture comprises adding dideoxynucleotide triphosphate terminated Cot-1 DNA to said amplification mixture.

12. The method of in-situ hybridization of claim 9 wherein said step of adding a repetitive sequence fraction to said amplification mixture comprises adding hybloc competitor DNA to said amplification mixture.

13. The method of in-situ hybridization of claim 9 wherein said hybridization mixture is a FISH hybridization mixture.

14. The method of in-situ hybridization of claim 9 wherein said hybridization mixture is an M-FISH hybridization mixture.

15. The method of in-situ hybridization of claim 9 wherein said hybridization mixture is a SKY hybridization mixture.

16. The method of in-situ hybridization of claim 9 wherein said hybridization mixture is a CGH hybridization mixture.

17. The method of in-situ hybridization of claim 9 wherein blocking DNA is not used in subsequent amplification mixtures.

18. The method of in-situ hybridization of claim 9 wherein said step of adding a repetitive sequence fraction DNA to said amplification mixture comprises using a standard PCR amplification procedure with the addition of 1 mg of species-specific Cot-1 DNA in said amplification mixture.


SEQUENCE LISTING (1 sequence)

SEQ ID NO: 1 - DNA, 22 bp, Artificial Sequence
  Description of Artificial Sequence Synthetic primer
ccgactcgagnnnnnnatgtgg